(19) United States
(12) Patent Application Publication
(10) Pub. No.: US 2013/0053983 A1
(43) Pub. Date: Feb. 28, 2013

(54) Safe Artificial Joint

(71) Applicant: Roee Lifshitz, Beit Hilel (IL)

(72) Inventor: Roee Lifshitz, Beit Hilel (IL)

(73) Assignee: Achiam LIFSHITZ, Beit Hilel (IL)

(21) Appl. No.: 13/642,950

(22) PCT Filed: May 4, 2011
(86) PCT No.: PCT/IL2011/000358

§ 371 Date: Oct. 25, 2012

Related U.S. Application Data
(60) Provisional application No. 61/330,972, filed on May 4, 2010.
(60) Provisional application No. 61/371,705, filed on Aug. 8, 2010.

(51) Int. Cl. A61F 2/64

(52) U.S. Cl. 623/43

(57) ABSTRACT

A joint for an artificial limb including a first support pivotally connected to a second support and a braking mechanism for limiting angular displacement between the first support and the second support actuatable when a time derivative of the angular displacement exceeds a predetermined value.

### FIELD OF THE INVENTION

The present invention relates to an artificial joint for a limb, and, more specifically, a joint provided with a braking mechanism for preventing sudden flexion of the joint.

### BACKGROUND OF THE INVENTION

The “instant centre”, or more properly the “instantaneous centre of zero relative velocity”, is a point where, for a very small change in the angle of knee flexion, the thigh section rotates about a point on an extension of the shank which appears to be temporarily fixed. For small angles of relative rotation one could imagine a temporary hinge connecting the shank and thigh sections at the instant centre. For larger angles of rotation the instant centre will change its location and a new temporary hinge must be imagined.

For a four-bar linkage knee, the instant centre (in any position of knee flexion) can always be located at the intersection of the centre lines of the anterior and posterior links which connect the socket section to the shank section of the prosthesis. As the knee flexion angle is increased the instant centre takes a series of positions which typically trace a path on an extension of the shank which progresses forward and downward toward the cosmetic or anatomical knee centre.

An elevated and posterior location of the instant centre will increase knee stability. With a single axis knee, the location of the knee joint is also dictated by placing it at an approximate anatomical location with good cosmetic appearance while seated with the knee at 90 degrees of flexion. A properly designed four-bar linkage knee also allows the possibility of locating the instant centre in full extension in a position within the desired stable region of the stability diagram, yet which maintains acceptable cosmetic appearance at 90 degrees of flexion.

U.S. Pat. No. 4,064,569 discloses an artificial polycentric knee-joint mechanism comprising a four-bar linkage arranged to provide a motion similar to a normal knee having positive stability during ambulation. The four bars include a knee-mounting block mounted to the stump-receiving socket member and a lower linkage support-carriage member affixed to the lower limb component, the mounting block and linkage support being operably interconnected by side bar links, one of which is tension controlled. Also, included is a braking control to insure normal swing phase movement of the artificial leg and a shock absorbing device to prevent excess terminal impact when the lower limb returns to the extended position of the leg prosthesis.

U.S. Pat. No. 4,090,264 ('264) discloses an artificial leg device including a double-jointed knee connection having a pair of tie arms pivotably connected to upper and lower support members and a pair of inner linkage arms pivotably connected to the upper and lower support members intermediate the tie arms. A gear drive assembly is connected between the upper and lower support members providing synchronous rotation therebetween. A ratchet coupling is connected to the gear drive assembly having a pivotable pawl member cooperating with a ratchet wheel to form a lock therebetween for locking the knee joint in a weight-supporting configuration. An operator assembly is carried by the lower support member for operating the pawl and includes an operator rod responsive to pressure on the foot, a resilient biasing means carried by the operator rod, and a quick-release mechanism carried adjacent the upper portion of the operator rod for releasing the resilient biasing means to provide quick and positive disengagement of the pawl from the ratchet.

It should be emphasized that the artificial leg device taught in '264 is stance-controlled in a vertical position only because the pawl-and-ratchet mechanism is activated when an amputated person steps on the artificial leg. However, in non-vertical positions, the artificial leg is not stance-controlled. In other words, the artificial leg can be suddenly flexed during amputated person's ambulation when the artificial leg is in the non-vertical position. Thus, there is a long-felt and unmet need to provide a safe artificial limb in which the possibility of sudden flexing of the artificial limb is precluded.

### SUMMARY OF THE INVENTION

It is hence one object of the invention to disclose a joint for preventing a sudden flexing of an artificial limb. The joint includes a first support pivotally connected to a second support and a braking mechanism for limiting angular displacement between the first support and the second support actuatable when a time derivative of the angular displacement exceeds a predetermined value.

Another object of the invention is to disclose the time derivative which is an angular velocity.

A further object of the invention is to disclose the predetermined value of the angular velocity ranging between about 1 and about 100 rad/sec.

A further object of the invention is to disclose the predetermined value of the angular velocity ranging between about 1 and about 10 rad/sec.

A further object of the invention is to disclose the time derivative which is an angular acceleration.

A further object of the invention is to disclose the predetermined value of the angular acceleration ranging between about 1 and about 2,000 rad/sec².

A further object of the invention is to disclose the predetermined value of the angular acceleration ranging between about 1 and about 100 rad/sec².

A further object of the invention is to disclose the first support pivotally connected to the second support via a linkage assembly.

A further object of the invention is to disclose the joint which is a knee joint having a thigh support and a shin support.

A further object of the invention is to disclose the linkage assembly selected from the group consisting of a single axis linkage, a polycentric linkage, a four-bar linkage, a hydraulic linkage, computer controlled linkage and any combination thereof.

A further object of the invention is to disclose the braking mechanism including (a) a pawl-and-ratchet mechanism; and (b) a triggering mechanism adapted for activating the pawl-and-ratchet mechanism.

A further object of the invention is to disclose the triggering mechanism including the pawl supported on a frame movable between a locking position in engagement with a tooth of the ratchet wheel so that the angular displacement of the bar means is prevented and a non-locking position allowing the ratchet wheel coupled to the bar means to be angularly displaced.

A further object of the invention is to disclose the triggering mechanism including an inertia mechanism responsive to the predetermined value of the time derivative.

A further object of the invention is to disclose the pawl movable between locked and unlocked positions against a bias of a spring.

A further object of the invention is to disclose the pawl angularly displaceable between the locked and unlocked positions.

A further object of the invention is to disclose the pawl linearly displaceable between the locked and unlocked positions.

A further object of the invention is to disclose the inertial mechanism including an off-axis member connected to a bar and fulcrum and a rotatable locking actuator adapted for placing the pawl into engagement with the ratchet; the off-axis member is designed for off-axis deviation and meshing with the locking actuator when the predetermined value of the time derivative of the angular displacement exceeds the predetermined value thereby.

A further object of the invention is to disclose the inertial mechanism including a disc member centrally disposed on a bar and fulcrum and a sprung lever pivotable at an off-axis point of the disc; the sprung lever is designed for centrifugal deviation and linear displacement of the pawl into the locked position when the time derivative of the angular displacement exceeds the predetermined value.

A further object of the invention is to disclose the braking mechanism including an electromechanical converter adapted for transmitting an electrical signal corresponding to the derivative and a triggering circuit adapted to activate the braking mechanism in response to the electrical signal corresponding to a predetermined value of the time derivative of the angular displacement.

A further object of the invention is to disclose the joint configured for assisting ambulation of an individual suffering from joint instability.

A further object of the invention is to disclose the first support and the second support configured for attachment to the limb of the individual.

A further object of the invention is to disclose the joint instability caused by a disorder selected from the group consisting of broken bones, arthritic joints, bowleg, knock-knee, knee hyperextension, muscular weakness, paralysis the any combination thereof.

A further object of the invention is to disclose the joint further comprising a derivative sensor and a micro-controller, said sensor is adapted for transmitting a signal corresponding to instantaneous values of derivatives to said micro-controller which triggers the braking mechanism when the derivatives exceeds the predetermined value of the time derivative of the angular displacement.

A further object of the invention is to disclose the joint further comprising a retarding mechanism adapted for providing a requisite torque for bending the joint.

A further object of the invention is to disclose the retarding mechanism including: (a) a casing accommodating a fluid; (b) a wiper mechanically connected to the limb, the wiper arranged to be angularly displaced within the casing; and (c) a valve openable in response to a predetermined value of fluid pressure.

A further object of the invention is to disclose the braking mechanism adapted to limit angular displacement of between said first support and said second support in response to a predetermined value of said fluid pressure.

A further object of the invention is to disclose the joint further comprising a transmission.

A further object of the invention is to disclose the transmission which is an epicyclical train transmission.

A further object of the invention is to disclose the braking mechanism including (a) an outer annular member provided at an inner surface thereof with a plurality of cogs circumferentially distributed over the surface; and (b) a inner rotatable member placed into said outer member provided with a plurality of pawls centrifugally engageable with the cogs of the outer member.

A further object of the invention is to disclose the joint further comprising an angular shock-absorbing mechanism.

A further object of the invention is to disclose the angular shock-absorbing mechanism including a first disc and a second disc rotatably interconnected to each other; the first disc is provided with a plurality of pins circumferentially distributed over the first disc; the second disc is provided with a plurality of circumferentially oriented slots configured for receiving the pins; inner walls of the slots are fringed with a rubber absorbing layer.

A further object of the invention is to disclose the epicyclical train transmission having two inputs.

A further object of the invention is to disclose one input provided with an additional pawl-ratchet mechanism.

A further object of the invention is to disclose a method of securing a limb from sudden flexing. The method includes a step of attaching the joint of the present invention to a limb of a subject in need.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIGS. 1a and 1b are schematic views of a knee joint;

FIGS. 2a and lb are frontal and side schematic views of a four bar knee joint (prior art);

FIG. 3 is a schematic view of a knee joint provided with a braking mechanism;

FIGS. 4a and 4b are schematic diagrams of a braking mechanism in an open and lock positions, respectively;

FIGS. 5a, 5b and 5c are schematic diagrams of a braking mechanism provided with linearly displaceable pawl;

FIGS. 6a and 6b are schematic diagrams of a braking mechanism provided with a weighted pendulum;

FIGS. 7a to 7e are photographs depicting a metal-made prototype;

FIGS. 8a to 8d are photographs depicting a carbon ceramics-made prototype;

FIG. 9 is a schematic view of a fluid retarding mechanism;

FIG. 10 is a schematic isometric view of a knee joint provided with an epicyclical transmission;

FIG. 11 is an exploded isometric view of the knee joint shown in FIG. 10

FIG. 12 is a schematic isometric view of an epicyclical transmission;

FIG. 13 is an exploded isometric view of a braking mechanism provided with the angular shock absorbing mechanism: and

FIG. 14 is a schematic isometric view of an epicyclical transmission provided with two inputs.

### DETAILED DESCRIPTION OF THE INVENTION

The following description is provided, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a joint for an artificial limb and a method of using the same.

According to one aspect of the invention there is provided a joint for an artificial limb, including a first support pivotally connected to a second support and a braking mechanism for limiting angular displacement between the first support and the second support. The braking mechanism is actuatable when a time derivative of the angular displacement exceeds a predetermined value.

The phrase “time derivative” used herein refers to the change rate of the angular displacement. Accordingly, the time derivative can be either the angular velocity or the angular acceleration of the angular displacement.

In one embodiment, the angular velocity triggering the braking mechanism is between about 1 and about 100 rad/sec. In another embodiment the angular velocity is between about 1 and about 10 rad/sec.

In accordance with another embodiment, the angular acceleration triggering the braking mechanism is between about 1 and about 2,000 rad/sec². In another embodiment the angular acceleration is between about 1 and about 100 rad/sec².

As used herein the term “about” refers to ? 10%.

The joint of the present invention can be, but not limited to, a knee joint having a thigh support and a shin support. An advantage of the knee joint of the present invention is that it prevents a sudden flexion of the joint.

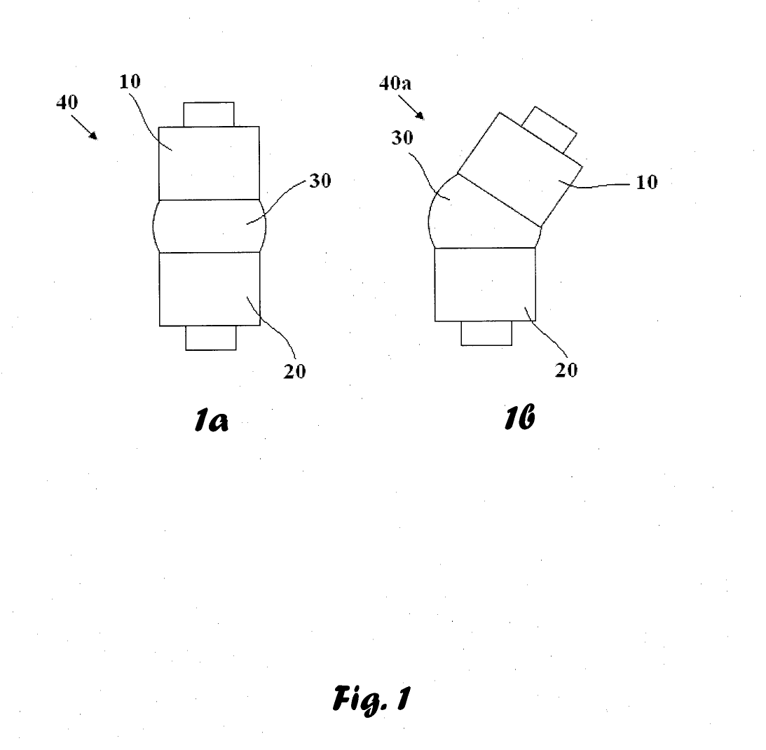

Reference is now made to FIGS 1a and 1b, showing a schematic diagram of an artificial knee joint according to the present invention in straightened and bent positions 40 and 40a, respectively. The joint 40/40a comprises thigh support 10, shin support 20 and linkage 30 pivotally interconnecting the aforesaid thigh support 10 and shin support 20 therebetween.

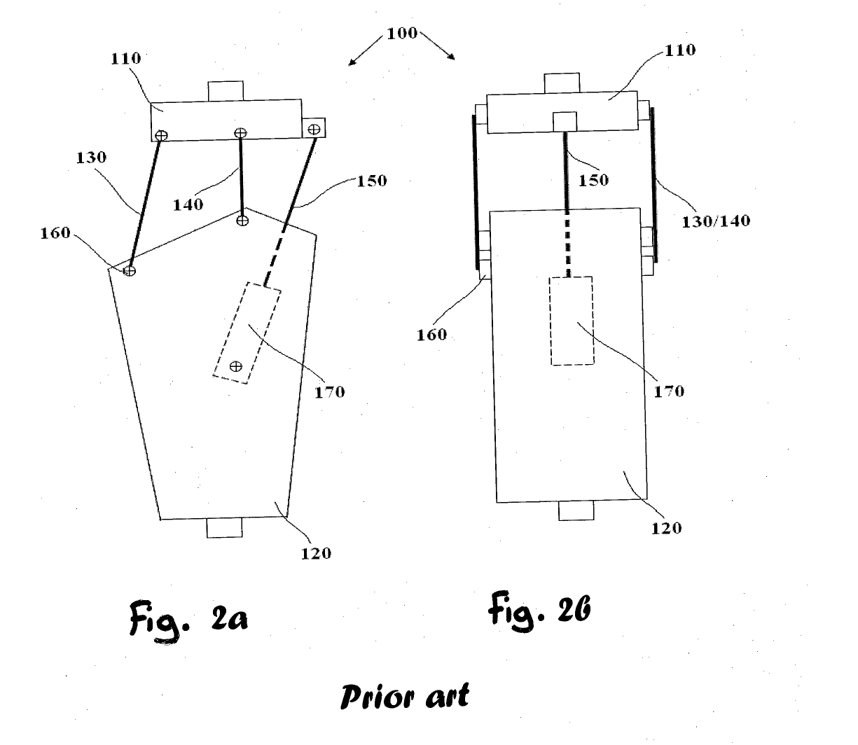

Reference is now made to FIGS. 2a and 2b, presenting frontal and side schematic views of the four bar knee joint 100 known in the art. The aforesaid joint 100 comprises a thigh support 110, shin support 120, a four bar linkage consisting of two pair of pivoted bars 130 and 140, a retarding mechanism 170 fixed to the shin means 120 and mechanically connected to thigh support 110 via a bar 150. The bars 130, 140 and 150 provided at terminals thereof with hinges 160. The four-bar linkage 130-140 enables the thigh support 110 to angularly displace. Specifically, in Fig. la the thigh support 110 declines clockwise in the drawing plane. In Fig. lb inclination is performed in the plane perpendicular to the drawing. The link support 110 and shin support 120 constitute articles mechanically connectable to thigh and shin portions of the artificial leg (not shown). The retarding mechanism 170 is mechanism adapted for providing a requisite torque when the join is bent. The retarding mechanism 170 improves prosthesis stability due to limiting a velocity of the relative angular displacement of the thigh and shin portions one relative to another. The retarding mechanism comprising a hydraulic cylinder is in the scope of the current invention. However, the aforesaid knee joint does not prevent from sudden flexion during amputated person's ambulation on the artificial leg.

According to one embodiment of the present invention the knee joint includes a braking mechanism comprising a pawl and ratchet mechanism and a triggering mechanism adapted for activating the pawl and ratchet mechanism. Optionally, the triggering mechanism includes an inertia locking mechanism responsive to the predetermined value of the time derivative. Suitable inertia-responsive lock mechanisms are well known in the art.

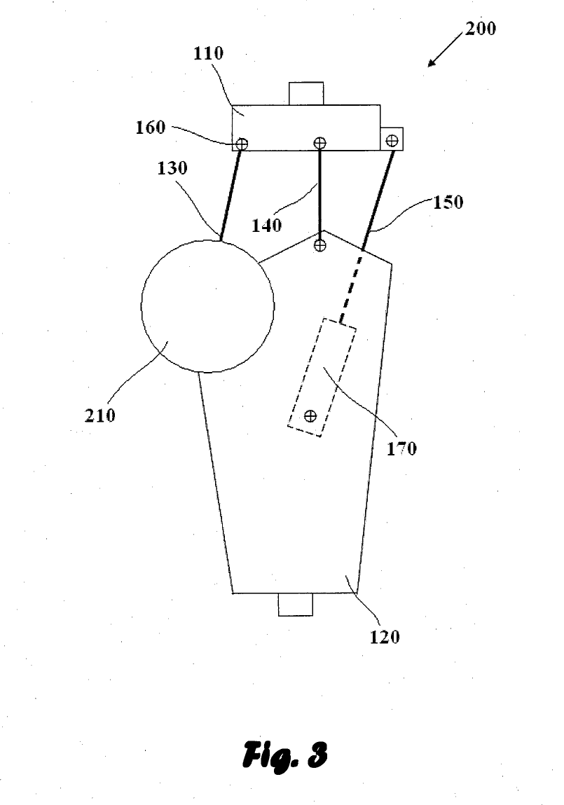

Reference is now made to FIG. 3, presenting an improved knee joint in accordance with the present invention. Knee joint 200 is provided with a braking mechanism 210. At least one bar means (for example, 130) is coupled to at least one link means (for example, 120) through the braking mechanism 210 such that the mechanism stops an angular displacement of the bar means relative to the link means responsively to exceeding a predetermined value of an time derivative of the angular displacement. The mechanism 210 prevents the amputated person from sudden fall because the mechanism 210 blocks the angular displacement of the bar means. According to an alternative embodiment of the current invention, the braking mechanism 210 comprises an electromechanical converter adapted for transmitting an electrical signal corresponding to the time derivative of the angular displacement and a triggering circuit adapted to activate the braking mechanism in response to the electrical signal responding to a predetermined value of the time derivative.

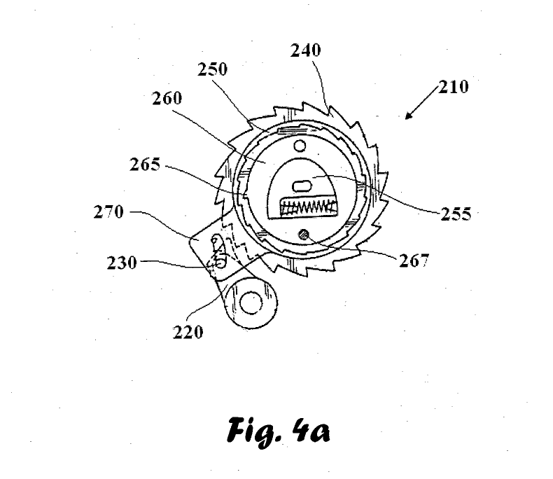

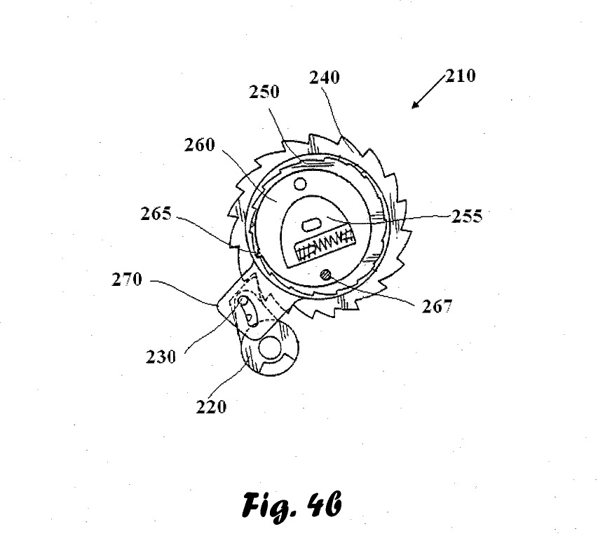

Reference is now made to FIGS. 4a and 4b, presenting a mechanical arrangement of the mechanism 210. A rotatable member 255 and a ratchet wheel 240 are mechanically connected to the bar means 130 (not shown). When the bar 130 is angularly displaced, the rotatable member 255 is rotated, as well. An inertial member 260 is loosely pivoted with a fulcrum 267. A frame 270 having an internal scalloped surface 250 is mechanically linked with a pawl 220 via a pin 230. When the rotatable member 255 mechanically connected to the bar 130 is accelerated up to a predetermined time derivative the inertial member 260 rotates around the fulcrum 267. The inertial member 260 is provided with a cog 265 which engages with internal scalloped surface 250. The frame 270 angularly moves the pawl 220 to the ratchet wheel 240 so that the pawl 220 locks the ratchet wheel 240. In other words, the sudden flexion of the knee joint (not shown) is prevented.

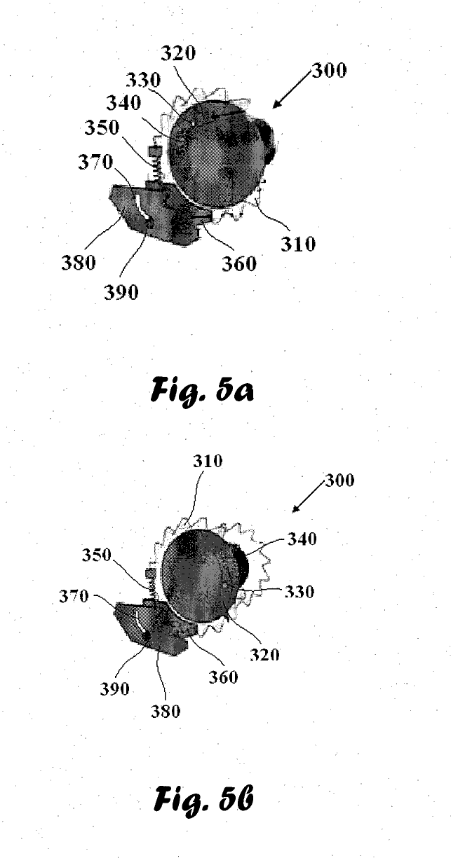

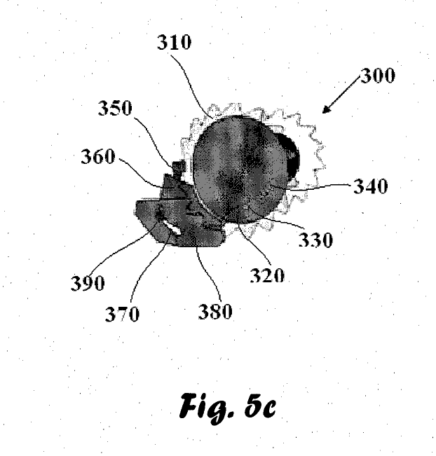

Reference is now made to FIGS. 5a to 5c, presenting an alternative embodiment 300 of the pawl-and-ratchet mechanism which comprises a ratchet wheel 310 and a pawl 360. The ratchet 310 is provided with a clutch lever 320 pivoted with a fulcrum 330 and sprung by a spring 340. The pawl 360 is disposed on a linearly movable frame 380 provided with a linear slot 370 accommodating a pin 390. As seen in FIG. 5b, at the predetermined value of the time derivative of the ratchet wheel 310, the clutch lever 320 deviates under action of a centrifugal force which becomes greater than a force of the spring 340. Then, the clutch lever 320 linearly displaces the frame 380 so that the pawl 360 comes into mesh with the ratchet wheel 310 and pawl-and-ratchet mechanism is locked (see FIG. 5c).

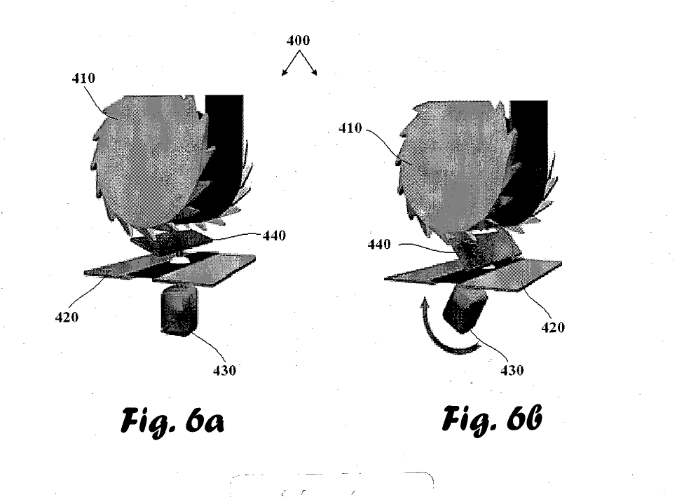

Reference is now made to FIGS. 6a and 6b, presenting an alternative embodiment 400 of the pawl-and-ratchet mechanism which comprises a ratchet wheel 410 and a pawl plate 440. The mechanism 400 locks the ratchet wheel 410 when rapidly decelerated. The central operating element in this mechanism is a weighted pendulum 430 mechanically connected to the pawl plate 440. The inertia causes the pendulum 430 to swing relative to a base plate 420. The pawl plate 440 on the other end of the pendulum catches hold of a toothed ratchet wheel 410.

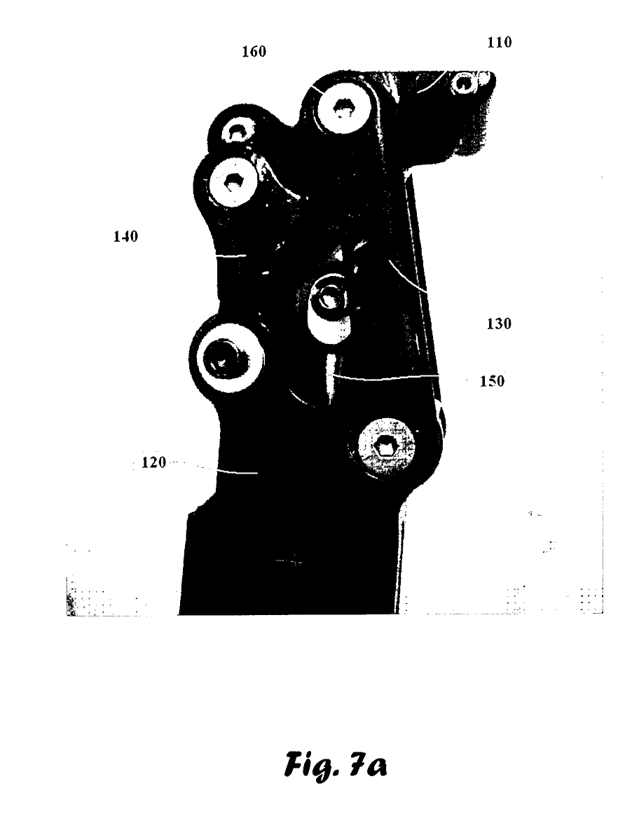

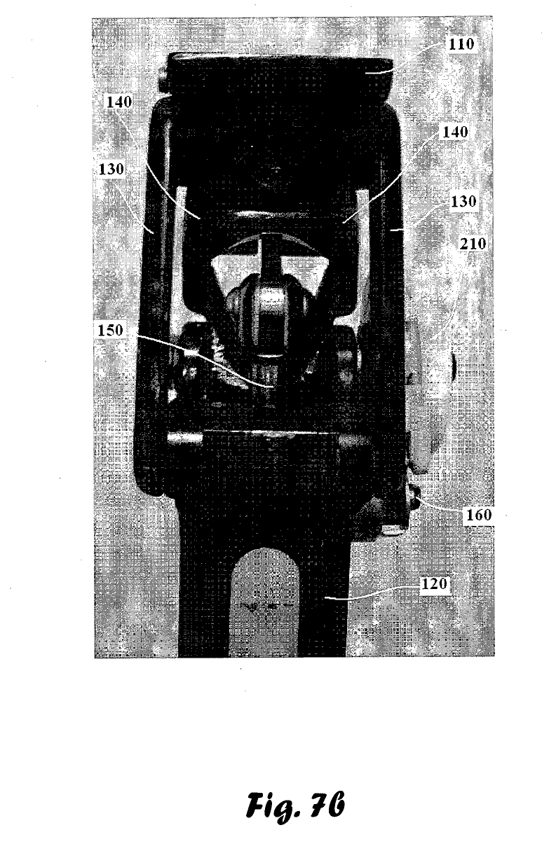

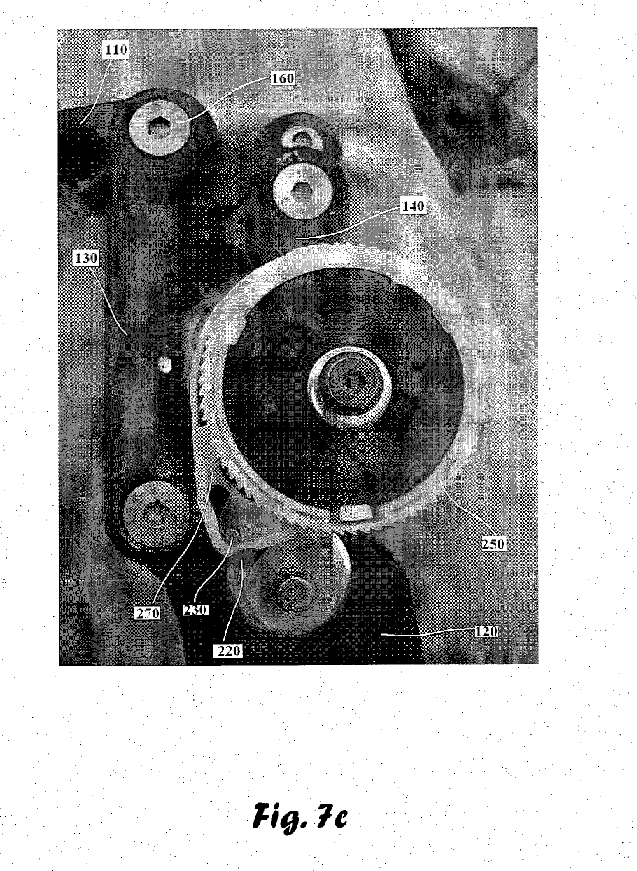

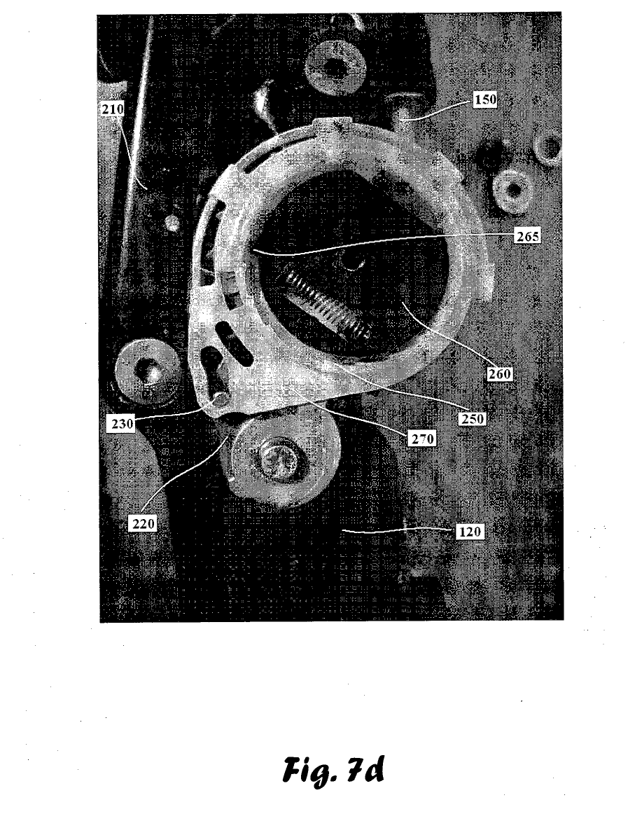

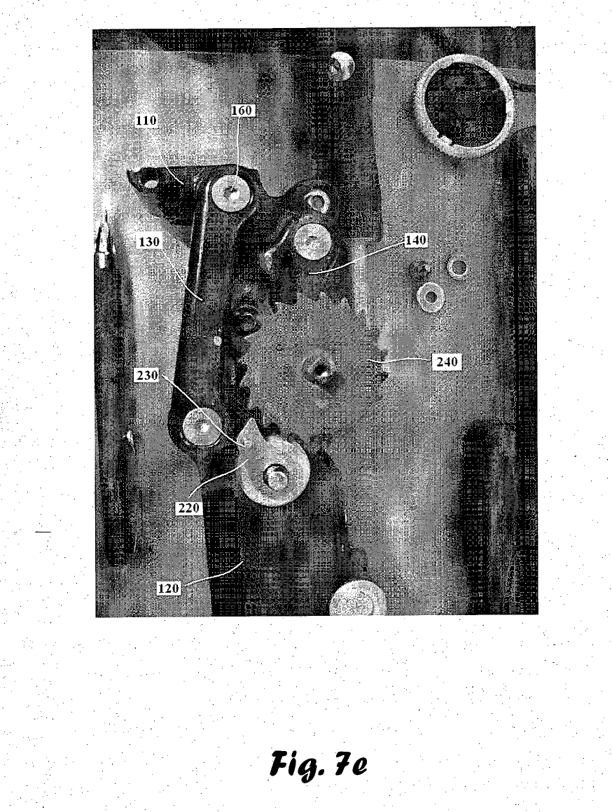

Reference is now made to FIGS. 7a to 7e, presenting photographs of the prototype made prove operability of the present invention. FIG. 7a shows a commercially available four bar knee joint, comprises a member 110 for fixing an upper (thigh) portion of the artificial leg, a member 120 for fixing a lower (shin) portion of the artificial leg, a front pair of bars 130, a back pair of bars 140, a link interconnecting a hydraulic cylinder (not shown) and member 110. The bars 130, 140 and 150 are pivoted by means of hinges 160. As shown in FIG. 7b, the pawl-and-ratchet mechanism 210 is mounted on the knee joint such that ratchet wheel is mechanically connected to the bar 130 and the pawl to the member 120. FIG. 7c frontally shows the pawl-and-ratchet mechanism covered by a cap 250. Referring to FIG. 7c, the pawl 250 is provided with a pin 230 accommodated in a frame 270. As explained above, if a member 260 rotates counter wise, under action of the inertial force, a member 260 loosely pivoted with a fulcrum (not shown) centrifugally deviates and a cog 265 thereof comes into mesh with a scalloped surface 250 (FIG. 7d). Then, rotational motion is transferred to the frame 250 which bring the pawl 220 into mesh with the ratchet wheel (not shown). FIG. 7e shows a pawl-ratchet pair which is adapted to stop sudden flexion of the knee joint.

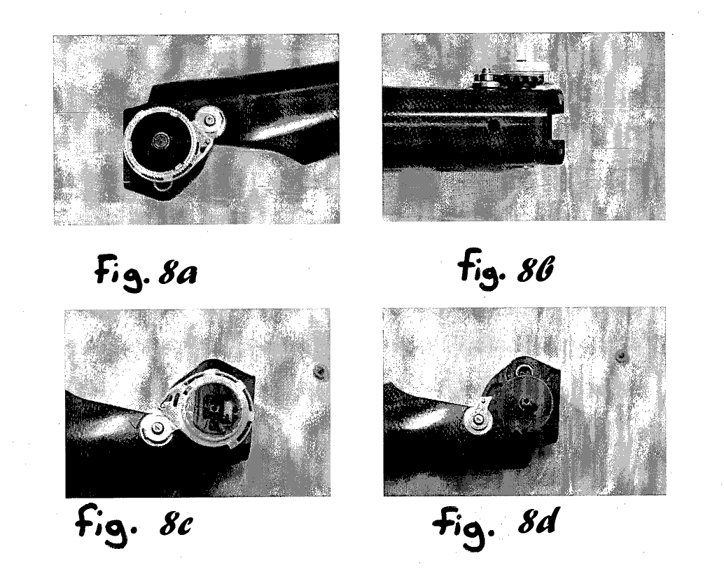

Reference is now made to FIGS. 8a to 8d, presenting the prototype made of carbon ceramics.

In another embodiment of the present invention the joint includes a retarding mechanism adapted for providing a requisite torque for bending the joint. Optionally, the retarding mechanism includes: (i) a casing accommodating a fluid, (ii) a wiper mechanically connected to the limb and arranged to be angularly displaced within the casing and (iii) a valve operable in response to a predetermined value of fluid pressure.

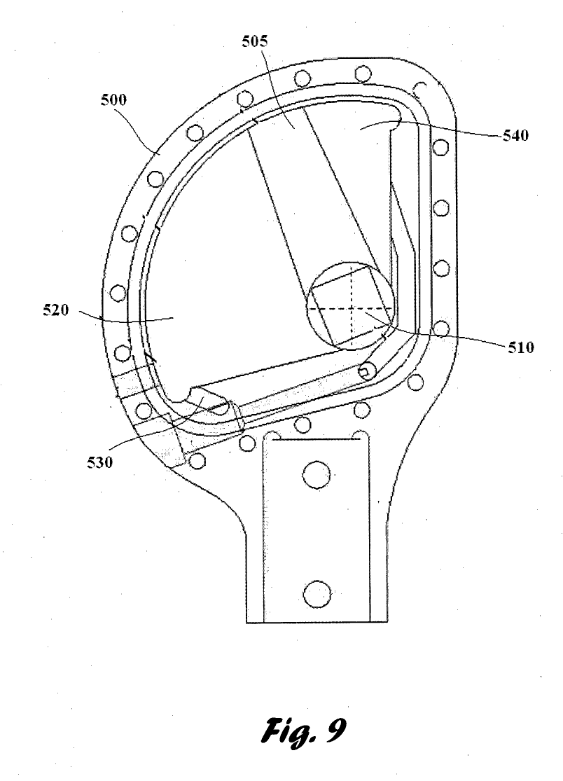

Reference is now made to FIG. 9, presenting an exemplary retarding mechanism. A wiper 505 is disposed into a casing. As the knee flexes, the arm 510 rotates counter-clockwise, the wiper 505 decreases the flexion chamber volume 520. Fluid in the flexion chamber 520 is forced through the valve channel 530, into the expanding extension chamber 540. The braking mechanism is adapted to stop angular displacement of the thigh support and shin support in response to a predetermined value of the fluid pressure.

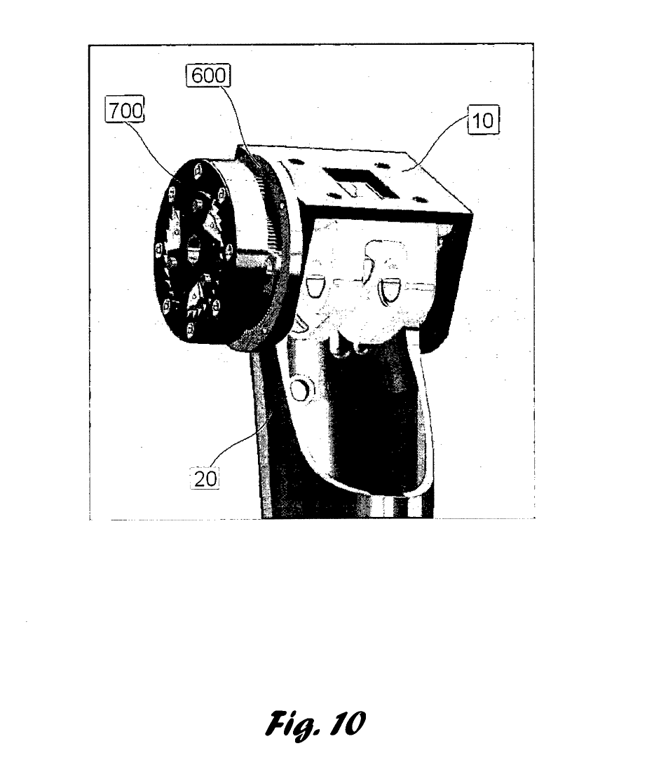

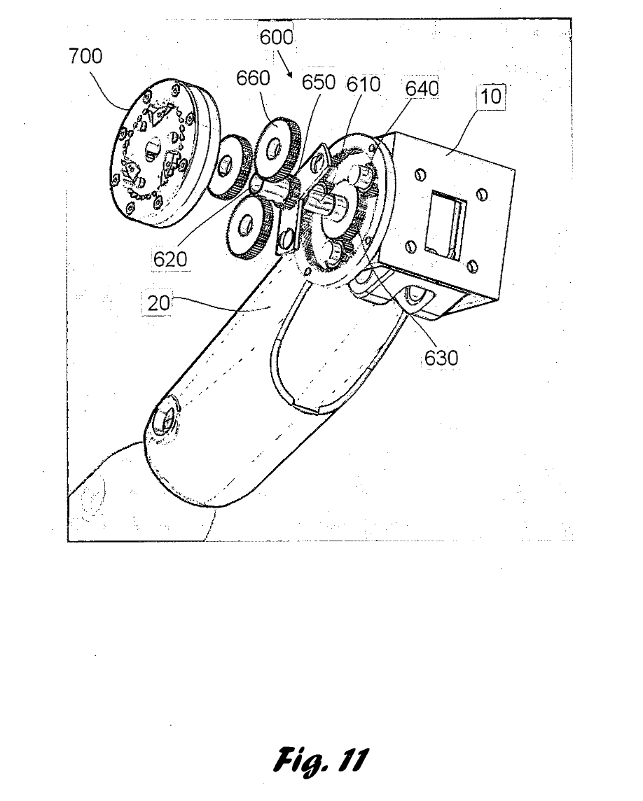

Reference is now made to FIGS. 10 and 11, presenting a joint provided with a cogwheel transmission. A purpose achievable by means of the transmission is to improve sensitivity of the braking mechanism. A derived value of angular displacement triggers the braking mechanism which is determined by mechanical parameters of the braking mechanism. The inserted transmission multiply increases the provided angular displacement and thus improves the sensitivity of the braking mechanisms. In accordance with the current embodiment of the invention, the joint comprises an epicyclical train 600 and a braking mechanism 700 (as specified below). The epicyclical train 600 transfers to the braking mechanism 700 a multiplied angular displacement between members 10 and 20.

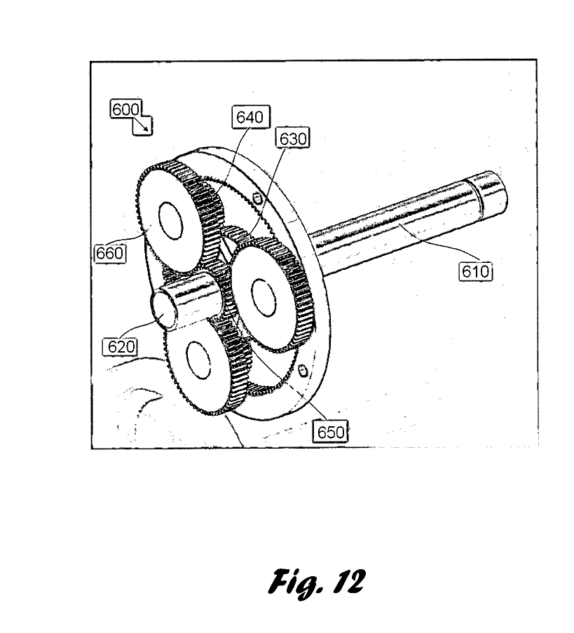

Reference is now made to FIG. 12, presenting an exemplary epicyclical train 600. The foresaid epicyclical train 600 comprises an input shaft 610, an output shaft 620 and cogwheels 630-660. A cogwheel 630 is disposed on the input shaft 610 and is in engagement with a cogwheel 640. The cogwheel 640 is mechanically interconnected with a cogwheel 660 which is in turn in engagement with a cogwheel 650 disposed on the output shaft 620. Thus, the angular displacement provided at the output shaft 620 is multiplied. In accordance with a preferred embodiment of the current invention, the transmission ratio is preferably 1:1, preferably 1:2, preferably 1:3, preferably 1:4, preferably 1:5, preferably 1:6, preferably 1:7, preferably 1:8, preferably 1:9, preferably 1:10.

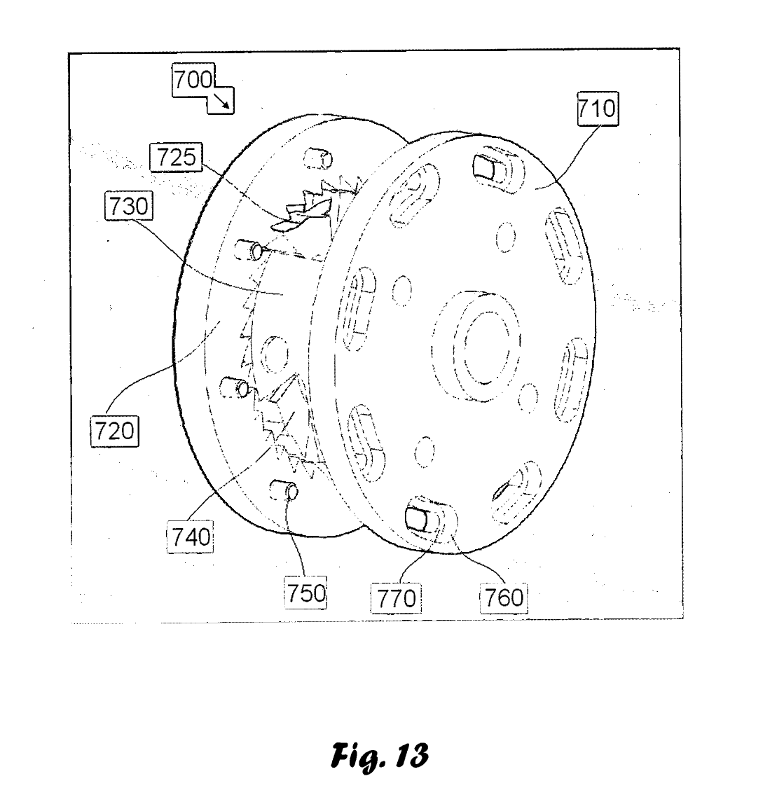

Reference is now made to FIG. 13, presenting a braking mechanism 700. The aforesaid mechanism 700 comprises two portions. The first portion further comprises (a) an outer annular member 720 provided at an inner surface thereof with a plurality of cogs 725 circumferentially spread over the inner surface; and an inner member 730 rotatably placed into the outer member 720 provided with a plurality of pawls 740 centrifugally engageable with the cogs 725 of the outer member 720. Thus, when an angular displacement is applied to the braking mechanism, the pawls 740 are blocked with the outer member 720, and relative rotation between members 720 and 730 is stopped.

The second portion constitutes an angular shock-absorbing mechanism comprising a disc 710 rotatably interconnected with the outer member 720. As seen in FIG. 13, the outer member 720 is provided with a plurality of pins 750 circumferentially spread over the outer member 720. The disc 710 is provided with a plurality of circumferentially oriented slots 760 configured for receiving the pins 750. Inner walls of the slots 760 are fringed with a rubber absorbing layer 770. Thus shocking angular displacement applied to the pins 750 is absorbed by the aforesaid rubber absorbing layer 770.

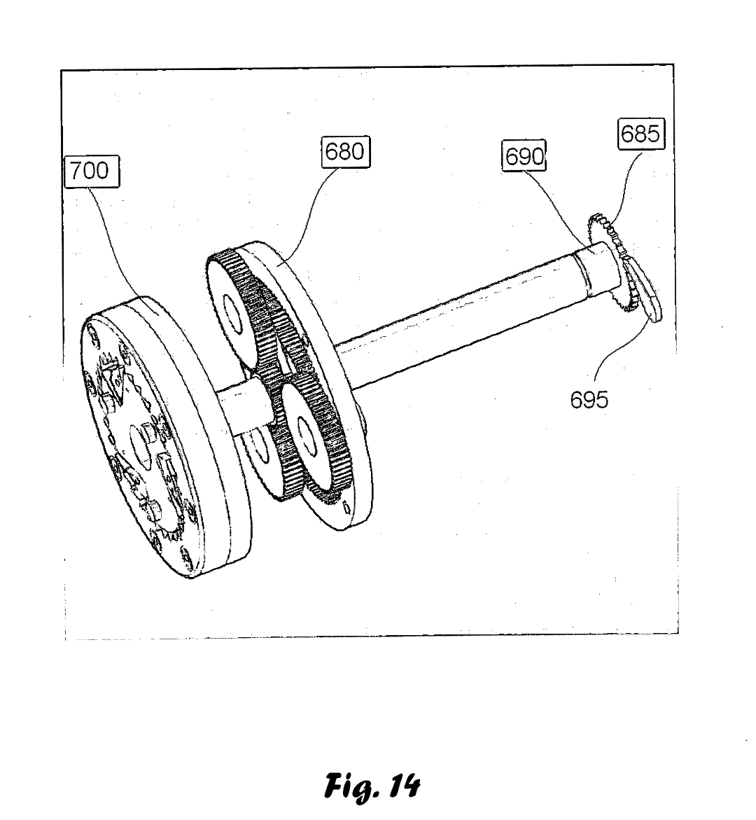

Reference is now made of FIG. 14, presenting an epicyclical train provided with two inputs 680 and 690. The braking mechanism 700 is mechanically interconnected with an additional pawl 695 engageable with an additional cogwheel 685. When time derivative of the angular displacement exceeds a predetermined value pawl 695 is triggered thereby locking the cogwheel. The purpose of the above is forcing the torque acting between load and gear to act between load to pawl 695 and cogwheel 685.

The knee joint of the present invention is used as follows: the knee joint is provided with upper and lower portions of the artificial leg so that the amputee can put (attach) the upper portion of the artificial leg on his/her amputated thigh. The lower portion of the artificial leg is adapted for providing a prop of the amputee on the ground. The upper and lower portions are mechanically connected to the knee joint. Then, the amputee puts the upper portion on the thigh stump and ambulates by means of stepping on the artificial leg.

In another aspect of the present invention there is provided a method of securing a limb from sudden flexing. The method includes a step of attaching the joint of the present invention to a limb of a subject in need. Preferably the joint is a knee joint attached to a leg stump of an amputee.

1-33. (canceled)

34. A joint for an artificial limb comprising a first support pivotally connected to a second support and a braking mechanism for limiting angular displacement between said first support and said second support actuatable when a time derivative of said angular displacement exceeds a predetermined value.

35. The joint according to claim 34, wherein said time derivative is selected from the group consisting of an angular velocity; angular acceleration and combination thereof

36. The joint of claim 34, wherein said first support is pivotally connected to said second support via a linkage assembly.

37. The joint according to claim 34, wherein the joint is a knee joint and further wherein said first support is a thigh support and said second support is a shin support.

38. The joint according to claim 34, wherein said braking mechanism comprises
a. a pawl-and-ratchet mechanism; and
b. a triggering mechanism adapted for activating said pawl-and-ratchet mechanism.

39. The joint according to claim 38, wherein said triggering mechanism comprises a pawl supported on a frame movable between a locking position in engagement with a tooth of a ratchet wheel so that said angular displacement of said braking mechanism is prevented and a non-locking position allowing said ratchet wheel to be angularly displaced.

40. The joint according to claim 38, wherein said triggering mechanism includes an inertia-locking mechanism responsive to said predetermined value of said time derivative.

41. The joint according to claim 38, wherein at least one of the following is true: said pawl is movable between locked and unlocked positions against a bias of a spring; and said pawl is angularly displaceable between said locked and unlocked positions.

42. The joint according to claim 41, wherein said pawl is linearly displaceable between said locked and unlocked positions.

43. The joint according to claim 40, wherein said inertia mechanism comprises an off-axis member connected to a bar and fulcrum and a rotatable locking actuator adapted for placing said pawl into engagement with said ratchet; said off-axis member is designed for off-axis deviation and meshing with said locking actuator when said predetermined value of said time derivative of said angular displacement exceeds said predetermined value thereby.

44. The joint according to claim 40, wherein said inertia mechanism comprises a disc member centrally disposed on a bar and fulcrum and a spring lever pivotable at an off-axis point of said disc; said sprung lever is designed for centrifugal deviation and linear displacement of said pawl into said locked position when said time derivative of said angular displacement exceeds said predetermined value.

45. The joint according to claim 34, wherein said braking mechanism comprises an electromechanical converter adapted for transmitting an electrical signal corresponding to said derivative and a triggering circuit adapted to activate said braking mechanism in response to said electrical signal corresponding to a predetermined value of said time derivative of said angular displacement.

46. The joint according to claim 45, wherein said first support and said second support are configured for attachment to the limb of the individual.

47. The joint according to claim 34, further comprising a derivative sensor and a micro-controller , said sensor is adapted for transmitting a signal corresponding to instantaneous values of derivatives to said micro-controller which triggers said braking mechanism when said derivatives exceeds said predetermined value of said time derivative of said angular displacement.

48. The joint according to claim 34, further comprising a retarding mechanism adapted for providing a requisite torque for bending said joint.

49. The joint of claim 48, wherein said retarding mechanism includes:
a. a casing accommodating a fluid;
b. a wiper mechanically connected to the limb, said wiper arranged to be angularly displaced within said casing;
c. a valve openable in response to a predetermined value of fluid pressure.

50. The joint according to claim 49, wherein said braking mechanism is adapted to limit angular displacement of between said first support and said second support in response to a predetermined value of said fluid pressure.

51. The joint according to claim 37 further comprising a transmission mechanism for transmitting the relative movements between said thigh support and said shin support; said transmission mechanism is an epicyclical train.

52. The joint according to claim 51, wherein said braking mechanism comprises
a. an outer annular member provided at an inner surface thereof with a plurality of cogs circumferentially distributed over said surface; and
b. an inner rotatable member placed into said outer member provided with a plurality of pawls centrifugally engageable with said cogs of said outer member.

53. The joint according to claim 34, further comprising an angular shock-absorbing mechanism; said angular shock-absorbing mechanism comprises a first disc and a second disc rotatably interconnected to each other; said first disc is provided with a plurality of pins circumferentially distributed over said first disc; said second disc is provided with a plurality of circumferentially oriented slots configured for receiving said pins; inner walls of said slots are fringed with a rubber absorbing layer.
